(19) United States
(12) Patent Application Publication
(10) Pub. No.: US 2025/0302675 A1
(43) Pub. Date: Oct. 2, 2025

(54) Flushable Removable Diaper Liner

(71) Applicant: Michelle B. Turner, Virginia Beach, VA (US)

(72) Inventor: Michelle B. Turner, Virginia Beach, VA (US)

(21) Appl. No.: 18/620,020

(22) Filed: Mar. 28, 2024

(51) Int. Cl. A61F 13/49; A61F 13/15; A61F 13/505; A61F 13/511

(52) U.S. Cl. CPC A61F 13/4906; A61F 13/5116; A61F 2013/15235; A61F 2013/5055

(57) ABSTRACT

The flushable removable diaper liner of the present invention is an easy-to-use diaper liner that can be removed from an existing diaper and flushed to enable a solid waste disposal device and method. The present invention makes it easy to dispose of solid waste in a flushing toilet, thus preventing solid waste entry into the landfill waste stream. Further, this invention reduces carbon emissions by preventing the solid waste from going to landfills. The device and method of the present invention can be in the form of an addition to an existing diaper and can be built/manufactured into an existing diaper by adding it to an existing manufacturing process.

### BACKGROUND OF THE INVENTION

### Technical Field

This invention relates generally to a flushable removable diaper liner. This invention relates more particularly to an apparatus/device for adding a flushable removable diaper liner to an existing diaper to enable disposal of solid waste in a flushing toilet instead of a landfill to generate carbon emissions.

This invention relates generally to apparatuses and devices for a diaper with a flushable removable diaper liner. This method also can be used with existing diapers. This invention relates more particularly to a flushable removable diaper liner to be added to an existing disposable or reusable diaper to enable disposal of solid waste in a flushing toilet instead of a landfill to generate carbon emissions.

### Background Art

Generally, today's disposable diapers include instructions on the side of the diaper package explaining that the used diaper with solid waste should shook or dunked in a toilet to get the poop off before disposing of the diaper. This is, of course, to prevent solid waste from entering the landfill waste stream. Solid waste in the landfill generates carbon emissions. People do not generally follow these instructions.

Instead, people throw those poopy diapers right into the trash and try to mask the smell with plastic bags, diaper pail, deodorizing sprays, and special powders. None of this changes the fact that we have poop sitting in the trash cans in our homes that eventually will go to the landfill. In the United States, there are current more than 18 billion diapers added to landfills every year.

Materials in landfills can seep into our groundwater, meaning that all that poop can seep into our groundwater. This is a serious public health and environmental concern.

There are liners available, but none of them are very user-friendly and easy to use. There is a not a flushable removable diaper liner that enables easy use to flush solid waste from used diapers.

In light of the foregoing prior art, there is a need for an easy to use flushable removable diaper liner to better dispose of solid waste in the flushing toilet thus preventing its entry into the landfill waste stream causing carbon emissions and potentially entering our groundwater.

### BRIEF SUMMARY OF THE INVENTION

The flushable removable diaper liner of the present invention makes it easy to dispose of solid waste in a flushing toilet, thus preventing solid waste entry into the landfill waste stream. Further, this invention reduces carbon emissions by preventing the solid waste from going to landfills. The device and method of the present invention can be in the form of an additional to an existing diaper. The device and method of the present invention can be built/manufactured into an existing diaper by adding it to an existing manufacturing process.

According to a first aspect of the invention there is a flushable removable diaper liner comprising an interior diaper shape made of a waste solid impermeable liquid permeable mesh in a break-away frame having a first tab configured for adding to an existing diaper for use in removing and flushing a solid waste prior to dis-positioning said existing diaper thus preventing said solid waste from entering a landfill.

According to a second aspect of the invention there is a flushable removable diaper liner comprising a second tab positioned an opposing end from said first tab configured to hold said flushable removable diaper liner to said existing diaper from an exterior of said existing diaper.

According to a third aspect of the invention there is a flushable removable diaper liner comprising an adhesive attachment means such as a hook and loop fabric, a sticky strip, or an adhesive strip configured for attachment to a diaper side of said flushable removable diaper liner.

According to a fourth aspect of the invention there is a flushable removable diaper liner wherein said interior diaper shape is fitted within a gusset edge of said existing diaper.

According to a fifth aspect of the invention there is a flushable removable diaper liner wherein said existing diaper is a disposable diaper or a reusable diaper.

According to a sixth aspect of the invention there is a flushable removable diaper liner wherein said first tab comprises an attachment means such as a hook and loop fabric, a sticky strip, or an adhesive strip configured for attachment to an external surface of said existing diaper.

According to a seventh aspect of the invention there is a flushable removable diaper liner wherein said first tab and/or said second tab comprises an attachment means such as a hook and loop fabric, a sticky strip, or an adhesive strip configured for attachment to an external surface of said existing diaper.

According to an eighth aspect of the invention there is a flushable removable diaper liner comprising a diaper having a liquid permeable liner, a liquid retaining liner, and a liquid impermeable exterior facing liner.

According to a ninth aspect of the invention there is a flushable removable diaper liner wherein said interior diaper shape is made from a biodegradable material, a compostable material, a bamboo material, and/or a cotton material.

According to a tenth aspect of the invention there is a removable diaper liner for a diaper comprising an additional top liner that can be removed and flushed without changing a structure or integrity of said diaper that will be thrown away wherein said additional top liner is a flushable removable diaper liner comprising an interior diaper shape made of a waste solid impermeable liquid permeable mesh in a break-away frame having a first tab configured for adding to an existing diaper for use in removing and flushing a solid waste prior to dis-positioning said existing diaper thus preventing said solid waste from entering a landfill, a liquid permeable liner, a liquid retaining liner, and a liquid impermeable exterior facing liner.

According to an eleventh aspect of the invention there is a removable diaper liner for a diaper comprising a second tab positioned an opposing end from said first tab configured to hold said flushable removable diaper liner to said existing diaper from an exterior of said existing diaper.

According to a twelfth aspect of the invention there is a removable diaper liner for a diaper comprising an adhesive attachment means such as a hook and loop fabric, a sticky strip, or an adhesive strip configured for attachment to a diaper side of said flushable removable diaper liner.

According to a thirteenth aspect of the invention there is a removable diaper liner for a diaper wherein said interior diaper shape is fitted within a gusset edge of said existing diaper.

According to a fourteenth aspect of the invention there is a removable diaper liner for a diaper wherein said existing diaper is a disposable diaper or a reusable diaper.

According to a fifteenth aspect of the invention there is a removable diaper liner for a diaper wherein said first tab comprises an attachment means such as a hook and loop fabric, a sticky strip, or an adhesive strip configured for attachment to an external surface of said existing diaper.

According to a sixteenth aspect of the invention there is a removable diaper liner for a diaper wherein said first tab and/or said second tab comprises an attachment means such as a hook and loop fabric, a sticky strip, or an adhesive strip configured for attachment to an external surface of said existing diaper.

According to a seventeenth aspect of the invention there is a removable diaper liner for a diaper wherein said interior diaper shape is made from a biodegradable material, a compostable material, a bamboo material, and/or a cotton material.

An advantage of the flushable diaper liner include: 1) allows for an easy way to flush diaper poop down the toilet to be processed by the waste management system, as is intended for human feces; 2) keeps poop out of garbage cans and from the smell permeating homes and other locations; 3) keeps poop out of landfills, where it can seep into and contaminate groundwater; 4) allows for a functional way to follow the instructions on the side of the box of most disposable diapers which says to shake poop into the toilet before throwing them away; 5) eliminates the need for plastic bags which are often used to wrap poopy diapers in to cover the smell; and 6) eliminates the need for special diaper pails, deodorizers, and other products used to cover the smell from throwing away diapers with poop still in them.

The invention will now be described, by way of example only, with reference to the accompanying drawings in which:

### BRIEF DESCRIPTION OF THE SEVERAL VIEWS OF THE DRAWINGS

FIG. 1 is an exploded view of the diaper liner being placed inside an existing diaper according to the invention; and

FIG. 2 is a perspective view of the diaper liner placed inside an existing diaper according to the invention.

### DETAILED DESCRIPTION OF THE INVENTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise,” or variations thereof such as “comprises” or “comprising,” will be understood to imply the inclusion of a stated element, integer or step, or group of elements integers or steps, but not the exclusion of any other element, integer or step, or group of elements, integers or steps.

Throughout this specification, the phrase “solid waste,” or variations thereof, will be understood to imply the feces, poop, and/or excrement deposited into diapers by the bodily functioning of a wearer of the diaper.

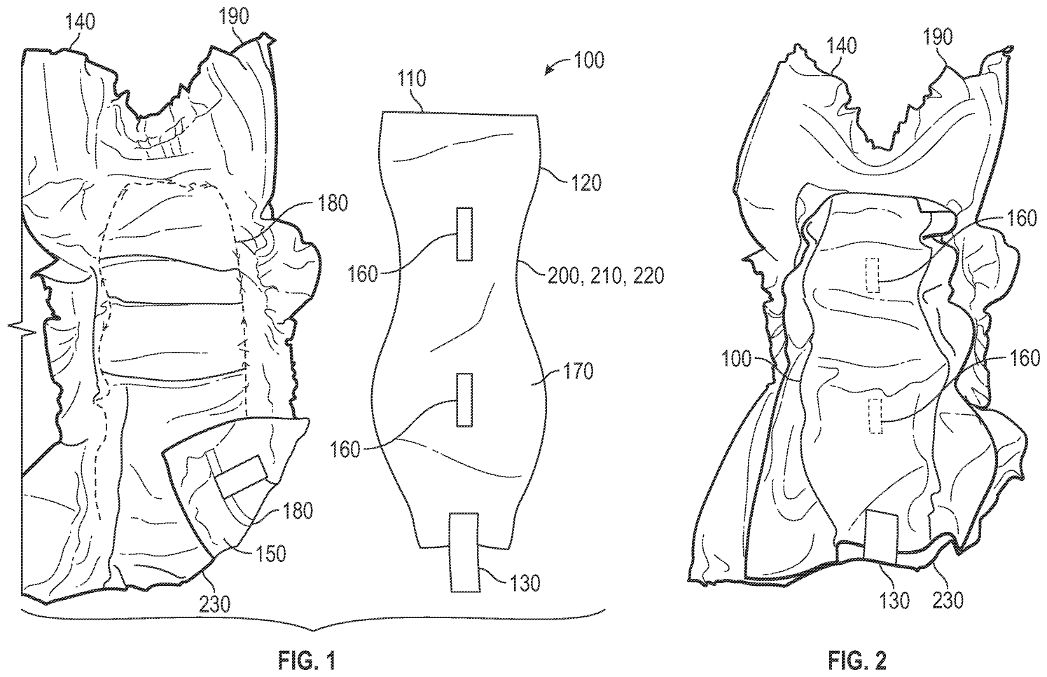

Index of Labelled Features in Figures. Features are listed in numeric order. Referring to the Figures, there is shown in FIGS. 1 and 2 the following features:

Element 100 which is a flushable removable diaper liner.

Element 110 which is an additional top liner.

Element 120 which is an interior diaper shape.

Element 130 which is a tab.

Element 140 which is an existing diaper.

Element 150 which is an exterior of the existing diaper.

Element 160 which is an adhesive attachment means.

Element 170 which is a diaper side of the flushable removable diaper liner

Element 180 which is a gusset edge of the existing diaper.

Element 190 which is a diaper.

Element 200 which is a liquid permeable liner.

Element 210 which is a liquid retaining liner.

Element 220 which is a liquid impermeable exterior facing liner.

Element 230 which is an external surface (exterior) of the existing diaper.

The diaper liner of the present invention can be manufactured separately from the diaper it will be used with. In this case, the flushable liner is added or installed into an existing diaper before application/use of the existing diaper.

The diaper liner of the present invention can be manufactured as a part of the diaper it will be used with. In this case, the flushable liner is already added or installed into an existing diaper before application/use of the existing diaper.

The present invention includes a diaper with an additional top liner that can be removed and flushed, without changing the structure or integrity of the remaining diaper that will be thrown away. There are several different options for detachment options and tabs to enable removal of solid waste by removing the top layer and flushing the inside. Removing the top layer does not impact the rest of the diaper.

The liner/mesh and the tabs, or attachment places/points that are part of the removable mesh/liner, are best made from flushable materials. Flushable materials are materials such as those used for creating plumbing safe toilet paper and wet wipes. Further, flushable materials are those that breakdown when immersed in liquid, such as water, so that they are safe for use in plumbing systems installed in homes and businesses.

The tabs, or attachment places/points that are made a part of the removable mesh/liner, are best placed on opposing ends or sides of the liner. At least one point of attachment is made to either the exterior or interior of the diaper that the liner is being used inside. Generally, it is best to use a pair (two) of attachment points.

In a first preferred embodiment of the present invention there is a flushable removable diaper liner comprising an interior diaper shape made of a waste solid impermeable liquid permeable mesh in a break-away frame having a first tab configured for adding to an existing diaper for use in removing and flushing a solid waste prior to dis-positioning said existing diaper thus preventing said solid waste from entering a landfill.

In an alternative embodiment of the first preferred embodiment of the present invention there is a flushable removable diaper liner comprising a second tab positioned an opposing end from said first tab configured to hold said flushable removable diaper liner to said existing diaper from an exterior of said existing diaper.

In an alternative embodiment of the first preferred embodiment of the present invention there is a flushable removable diaper liner comprising an adhesive attachment means such as a hook and loop fabric, a sticky strip, or an adhesive strip configured for attachment to a diaper side of said flushable removable diaper liner.

In an alternative embodiment of the first preferred embodiment of the present invention there is a flushable removable diaper liner wherein said interior diaper shape is fitted within a gusset edge of said existing diaper.

In an alternative embodiment of the first preferred embodiment of the present invention there is a flushable removable diaper liner wherein said existing diaper is a disposable diaper or a reusable diaper.

In an alternative embodiment of the first preferred embodiment of the present invention there is a flushable removable diaper liner wherein said first tab comprises an attachment means such as a hook and loop fabric, a sticky strip, or an adhesive strip configured for attachment to an external surface of said existing diaper.

In an alternative embodiment of the first preferred embodiment of the present invention there is a flushable removable diaper liner wherein said first tab and/or said second tab comprises an attachment means such as a hook and loop fabric, a sticky strip, or an adhesive strip configured for attachment to an external surface of said existing diaper.

In an alternative embodiment of the first preferred embodiment of the present invention there is a flushable removable diaper liner comprising a diaper having a liquid permeable liner, a liquid retaining liner, and a liquid impermeable exterior facing liner.

In an alternative embodiment of the first preferred embodiment of the present invention there is a flushable removable diaper liner wherein said interior diaper shape is made from a biodegradable material, a compostable material, a bamboo material, and/or a cotton material.

In a second preferred embodiment of the present invention there is a flushable removable diaper liner for a diaper comprising an additional top liner that can be removed and flushed without changing a structure or integrity of said diaper that will be thrown away wherein said additional top liner is a flushable removable diaper liner comprising an interior diaper shape made of a waste solid impermeable liquid permeable mesh in a break-away frame having a first tab configured for adding to an existing diaper for use in removing and flushing a solid waste prior to dis-positioning said existing diaper thus preventing said solid waste from entering a landfill, a liquid permeable liner, a liquid retaining liner, and a liquid impermeable exterior facing liner.

In an alternative embodiment of the second preferred embodiment of the present invention there is a flushable removable diaper liner comprising a second tab positioned an opposing end from said first tab configured to hold said flushable removable diaper liner to said existing diaper from an exterior of said existing diaper.

In an alternative embodiment of the second preferred embodiment of the present invention there is a flushable removable diaper liner comprising an adhesive attachment means such as a hook and loop fabric, a sticky strip, or an adhesive strip configured for attachment to a diaper side of said flushable removable diaper liner.

In an alternative embodiment of the second preferred embodiment of the present invention there is a flushable removable diaper liner wherein said interior diaper shape is fitted within a gusset edge of said existing diaper.

In an alternative embodiment of the second preferred embodiment of the present invention there is a flushable removable diaper liner wherein said existing diaper is a disposable diaper or a reusable diaper.

In an alternative embodiment of the second preferred embodiment of the present invention there is a flushable removable diaper liner wherein said first tab comprises an attachment means such as a hook and loop fabric, a sticky strip, or an adhesive strip configured for attachment to an external surface of said existing diaper.

In an alternative embodiment of the second preferred embodiment of the present invention there is a flushable removable diaper liner wherein said first tab and/or said second tab comprises an attachment means such as a hook and loop fabric, a sticky strip, or an adhesive strip configured for attachment to an external surface of said existing diaper.

In an alternative embodiment of the second preferred embodiment of the present invention there is a flushable removable diaper liner wherein said interior diaper shape is made from a biodegradable material, a compostable material, a bamboo material, and/or a cotton material.

### Advantages of a Flushable Diaper Liner:

Allows for an easy way to flush diaper poop down the toilet to be processed by the waste management system, as is intended for human feces. Flushable diaper liners provide an easy method to dispose of diaper waste by flushing it down the toilet. This aligns with the intended disposal method for human feces, allowing for efficient processing by the waste management system.

Keeps poop out of garbage cans and from the smell permeating homes and other locations. By keeping poop out of garbage cans, flushable diaper liners prevent the smell from permeating homes and other locations. This helps maintain a more pleasant and hygienic environment for both caregivers and infants.

Keeps poop out of landfills, where it can seep into and contaminate groundwater. By diverting poop away from landfills, flushable diaper liners help prevent the contamination of groundwater. This contributes to reducing environmental pollution and protecting ecosystems.

Allows for a functional way to follow the instructions on the side of the box of most disposable diapers which says to shake poop into the toilet before throwing them away. Many disposable diaper packages advise shaking poop into the toilet before disposal. Flushable liners provide a functional way to adhere to these instructions, ensuring proper waste disposal practices are followed.

Eliminates the need for plastic bags which are often used to wrap poopy diapers in to cover the smell. The use of flushable diaper liners eliminates the need for plastic bags typically used to wrap soiled diapers. This reduces plastic waste generation and its associated environmental impact.

Eliminates the need for special diaper pails, deodorizers, and other products used to cover the smell from throwing away diapers with poop still in them. Flushable diaper liners eliminate the need for special diaper pails, deodorizers, and other products used to manage the smell associated with throwing away diapers containing poop. This not only saves money for caregivers but also reduces reliance on additional consumables.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A flushable removable diaper liner comprising
an interior diaper shape made of a waste solid impermeable liquid permeable mesh in a break-away frame having a first tab configured for adding to an existing diaper for use in removing and flushing a solid waste prior to dis-positioning said existing diaper thus preventing said solid waste from entering a landfill.

2. The flushable removable diaper liner of claim 1 comprises a second tab positioned an opposing end from said first tab configured to hold said flushable removable diaper liner to said existing diaper from an exterior of said existing diaper.

3. The flushable removable diaper liner of claim 1 comprising an adhesive attachment means such as a hook and loop fabric, a sticky strip, or an adhesive strip configured for attachment to a diaper side of said flushable removable diaper liner.

4. The flushable removable diaper liner of claim 1 wherein said interior diaper shape is fitted within a gusset edge of said existing diaper.

5. The flushable removable diaper liner of claim 1 wherein said existing diaper is a disposable diaper or a reusable diaper.

6. The flushable removable diaper liner of claim 1 wherein said first tab comprises an attachment means such as a hook and loop fabric, a sticky strip, or an adhesive strip configured for attachment to an external surface of said existing diaper.

7. The flushable removable diaper liner of claim 2 wherein said first tab and/or said second tab comprises an attachment means such as a hook and loop fabric, a sticky strip, or an adhesive strip configured for attachment to an external surface of said existing diaper.

8. The flushable removable diaper liner of claim 1 comprising a diaper having
a liquid permeable liner,
a liquid retaining liner, and
a liquid impermeable exterior facing liner.

9. The flushable removable diaper liner of claim 1 wherein said interior diaper shape is made from a biodegradable material, a compostable material, a bamboo material, and/or a cotton material.

10. A removable diaper liner for a diaper comprising an additional top liner that can be removed and flushed without changing a structure or integrity of said diaper that will be thrown away
wherein said additional top liner is a flushable removable diaper liner comprising an interior diaper shape made of a waste solid impermeable liquid permeable mesh in a break-away frame having a first tab configured for adding to an existing diaper for use in removing and flushing a solid waste prior to dis-positioning said existing diaper thus preventing said solid waste from entering a landfill,
a liquid permeable liner,
a liquid retaining liner, and
a liquid impermeable exterior facing liner.

11. The flushable removable diaper liner of claim 10 comprises a second tab positioned an opposing end from said first tab configured to hold said flushable removable diaper liner to said existing diaper from an exterior of said existing diaper.

12. The flushable removable diaper liner of claim 10 comprising an adhesive attachment means such as a hook and loop fabric, a sticky strip, or an adhesive strip configured for attachment to a diaper side of said flushable removable diaper liner.

13. The removable diaper liner of claim 10 wherein said interior diaper shape is fitted within a gusset edge of said existing diaper.

14. The removable diaper liner of claim 10 wherein said existing diaper is a disposable diaper or a reusable diaper.

15. The flushable removable diaper liner of claim 10 wherein said first tab comprises an attachment means such as a hook and loop fabric, a sticky strip, or an adhesive strip configured for attachment to an external surface of said existing diaper.

16. The flushable removable diaper liner of claim 11 wherein said first tab and/or said second tab comprises an attachment means such as a hook and loop fabric, a sticky strip, or an adhesive strip configured for attachment to an external surface of said existing diaper.

17. The flushable removable diaper liner of claim 10 wherein said interior diaper shape is made from a biodegradable material, a compostable material, a bamboo material, and/or a cotton material.
